(19) United States
(12) Patent Application Publication
(10) Pub. No.: US 2015/0166435 A1
(43) Pub. Date: Jun. 18, 2015

(54) METHODS AND APPARATUSES FOR PROCESSING HYDROCARBONS

(71) Applicant: UOP LLC, Des Plaines, IL (US)

(72) Inventors: Manuela Serban, Glenview, IL (US); Gregory R. Werba, Arlington Heights, IL (US); Matthew Lippmann, Chicago, IL (US); Clayton Colin Sadler, Arlington Heights, IL (US); Rajeswar Gattupalli, Arlington Heights, IL (US)

(73) Assignee: UOP LLC, Des Plaines, IL (US)

(21) Appl. No.: 14/105,375

(22) Filed: Dec. 13, 2013

(51) Int. Cl. C07C 4/12

(52) U.S. Cl. CPC C07C 4/12

(57) ABSTRACT

Methods and apparatuses for processing hydrocarbons are provided. In one embodiment, a method for processing hydrocarbons includes fractionating a feed stock to form a C6-C10 naphtha stream and a C11⁺ hydrocarbon stream. The method reforms the C6-C10 naphtha stream. Further, the method cracks the C11⁺ hydrocarbon stream to form a stream of C6-C10 hydrocarbons and extracts aromatics from the stream of C6-C10 hydrocarbons to form an extract stream. The method includes combining the C6-C10 naphtha stream and the extract stream containing the aromatics. Also, the method includes processing the C6-C10 naphtha stream and the extract stream in an aromatics complex to form selected aromatic products. Further, the embodiment may include reforming raffinate streams.

### TECHNICAL FIELD

The technical field generally relates to apparatuses and methods for processing hydrocarbons, and more particularly relates to methods and apparatuses that produce aromatics.

### BACKGROUND

Aromatics, particularly benzene, toluene, ethylbenzene, and the xylenes (ortho, meta, and para isomers), which are commonly referred to as “BTEX” or more simply “BTX,” are extremely useful chemicals in the petrochemical industry. They represent the building blocks for materials such as polystyrene, styrene-butadiene rubber, polyethylene terephthalate, polyester, phthalic anhydride, solvents, polyurethane, benzoic acid, and numerous other components. Conventionally, BTEX is obtained for the petrochemical industry by separation and processing of fossil-fuel petroleum fractions, for example, in catalytic reforming or cracking refinery process units, followed by BTX recovery units.

Typically, integrated refining-petrochemical complexes separate a crude feedstock into a “straight run” or desired fraction of naphtha, such as C6-C10 naphtha, i.e., naphtha containing hydrocarbons having carbon chain lengths of six to ten, and a heavier fraction containing longer chain hydrocarbons such as heavy oils and residues. The naphtha stream typically undergoes reforming to produce a reformate with an increased aromatic content. The heavier fraction is typically cracked, such as by a fluid catalytic cracking (FCC) unit to form a “heart cut” or desired fraction of hydrocarbons, such as C6-C10 FCC hydrocarbons.

Conventionally, the naphtha stream and the FCC stream are processed to form selected aromatics. For example, a conventional process cracks the heavier fraction to form the FCC hydrocarbon stream and combines the FCC hydrocarbon stream with the straight run naphtha. Then, the combined stream is passed through a reforming unit to form a reformate. The reformate is processed in an aromatics complex to produce selected aromatic products, such as benzene and para-xylene.

Because aromatics are the building blocks of so many materials, there is a need to increase production of desired aromatics from integrated refining-petrochemical complexes. Typically, reforming units are used to produce aromatics from straight run naphtha, however such reforming units may convert existing aromatics in streams combined with straight run naphtha to other less desired compounds. Thus, there is a need to increase aromatics production without decreasing the value of other streams produced in the integrated refining-petroleum complexes, such as gasoline blends.

Accordingly, it is desirable to provide methods and apparatuses for processing hydrocarbons that produce aromatics. It is also desirable to provide methods and apparatuses for processing hydrocarbons that enable an increase in the production of aromatics through extracting of aromatics in a stream that bypasses a reforming unit. Also, it is desirable to provide such methods and apparatuses that operate economically. Furthermore, other desirable features and characteristics will become apparent from the subsequent detailed description and the appended claims, taken in conjunction with the accompanying drawings and the foregoing technical field and background.

### BRIEF SUMMARY

Methods and apparatuses for processing hydrocarbons are provided. In an exemplary embodiment, a method for processing hydrocarbons includes fractionating a feed stock to form a C6-C10 naphtha stream and a C11⁺ hydrocarbon stream. The method reforms the C6-C10 naphtha stream. Further, the method cracks the C11⁺ hydrocarbon stream to form a stream of C6-C10 hydrocarbons and extracts aromatics from the stream of C6-C10 hydrocarbons to form an extract stream. The method includes combining the C6-C10 naphtha stream and the extract stream containing the aromatics. Also, the method includes processing the C6-C10 naphtha stream and the extract stream in an aromatics complex to form selected aromatic products. Further, the embodiment may include reforming raffinate streams.

In another embodiment, a method for processing hydrocarbons includes fractionating a hydrocarbon stream in a fractionation unit and forming a first fraction and a second fraction. The method introduces the first fraction to a reforming unit and reforms the first fraction to form a reformate stream. The method includes feeding the reformate stream to an aromatics processing zone and producing a benzene product and a para-xylene product. Further, the method introduces the second fraction into a fluid catalytic cracking (FCC) unit and cracks the second fraction to form a cracked stream of hydrocarbons. The method includes feeding the cracked stream to an aromatic extraction unit and extracting aromatics from the cracked stream in an extract stream. The method further includes bypassing the reforming unit with the extract stream and introducing the extract stream to the aromatics processing zone, wherein the extract stream is processed in the aromatic processing zone to produce the benzene product and the para-xylene product.

In another embodiment, an apparatus for processing hydrocarbons is provided. The apparatus includes a fractionation unit configured to form a C6-C10 naphtha stream and a C11⁺ hydrocarbon stream from a feed stock. The apparatus further includes a cracking unit configured to crack the C11⁺hydrocarbon stream to form a stream of C6-C10 hydrocarbons and a first aromatic extraction unit configured to extract a first aromatic stream from the stream of C6-C10 hydrocarbons. Also, the apparatus includes a reforming unit configured to reform the C6-C10 naphtha stream and form a reformate and a second aromatic extraction unit configured to extract a second aromatic stream from the reformate. The apparatus is provided with an aromatics processing unit configured to produce a benzene product and a para-xylene product from the first aromatic stream and the second aromatic stream.

### BRIEF DESCRIPTION OF THE DRAWINGS

Embodiments of methods and apparatuses for processing hydrocarbons will hereinafter be described in conjunction with the following drawing figures wherein:

FIG. 1 is a schematic diagram of an apparatus and method for processing hydrocarbons in accordance with an embodiment; and

FIG. 2 is a schematic diagram of an apparatus and method for processing hydrocarbons in accordance with an alternate embodiment.

### DETAILED DESCRIPTION

The following detailed description is merely exemplary in nature and is not intended to limit the methods or apparatuses for processing hydrocarbons. Furthermore, there is no intention to be bound by any theory presented in the preceding background or the following detailed description.

Various embodiments of methods and apparatuses for processing hydrocarbons with enhanced production of valuable product streams are described herein. For example, embodiments herein provide for the enhanced production of aromatics, such as for example benzene, toluene, and xylene (BTX). The embodiments produce additional aromatics from FCC C6-C10 hydrocarbon streams as compared to conventional processing. Exemplary embodiments utilize aromatics recovery from the FCC C6-C10 hydrocarbon stream and avoid reforming those aromatics. In one embodiment, an extract stream including aromatics is removed from the FCC C6-C10 hydrocarbon stream and fed to an aromatics complex including fractionation and isomerization units to produce streams of desired aromatic species. In another embodiment, a portion of the extract stream including aromatics is removed from the FCC C6-C10 hydrocarbon stream and fed to the naphtha reformer. In another embodiment, the extraction of aromatics from the FCC C6-C10 hydrocarbon stream forms a raffinate comprising primarily paraffins and olefins that is fed to the naphtha reformer and an extract that is fed to an aromatics complex.

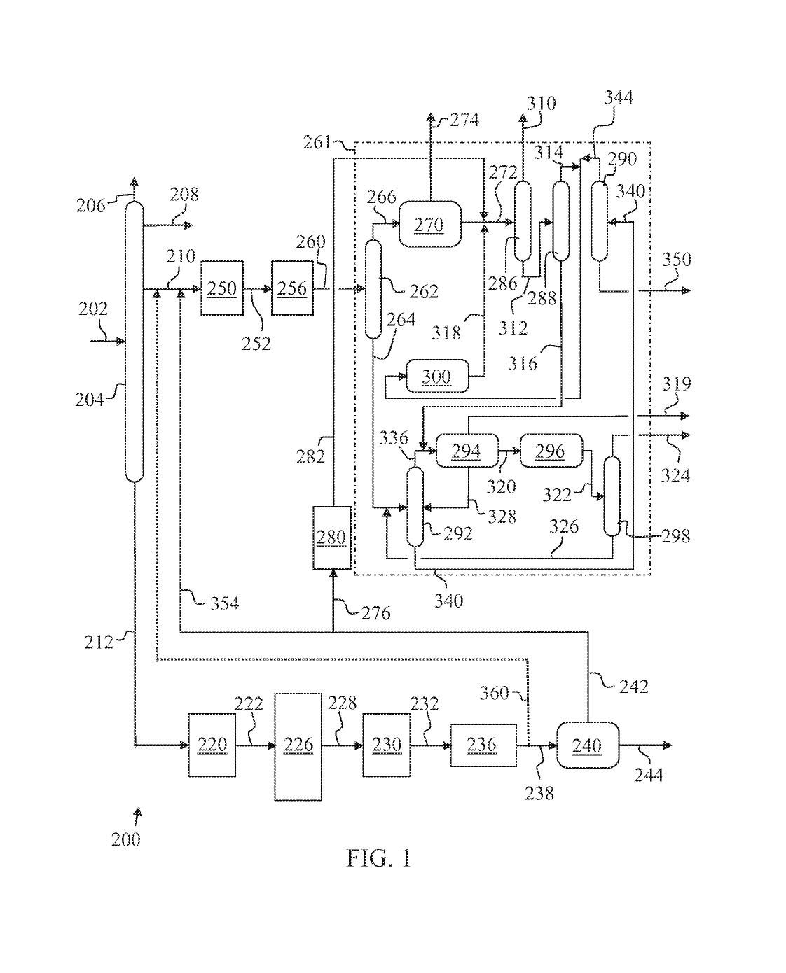

Referring to FIG. 1, an exemplary apparatus 200 is provided for processing hydrocarbons. As shown, a feedstock 202 is fed to the apparatus 200. An exemplary feedstock 202 is crude oil or may be other hydrocarbon streams. The feedstock 202 is fed to a crude distillation column 204 that fractionates the feedstock 202 into a stream 206, such an overhead stream, containing liquefied petroleum gas, a stream 208, such as an upper sidedraw stream, containing light naphtha such as naphtha containing hydrocarbons with carbon chains lengths of 5 or less, a stream 210, such as lower sidedraw stream, containing heavy or straight-run naphtha, for example C6-C10 naphtha (naphtha including hydrocarbons having carbon chain lengths of six to ten), and a stream 212, such as a bottom stream, containing C11⁺ hydrocarbon (hydrocarbons having carbon chain lengths of eleven or greater than eleven) such as heavy oils and residues.

In the exemplary embodiment, the stream 212 is processed by a residue hydrotreating unit 220 that removes sulfur, nitrogen, organometallics, and asphaltenes from the stream 212 to form a hydrotreated stream 222. The residue hydrotreating unit 220 may use a fixed-bed catalytic hydrotreating process with catalysts employed to facilitate demetallization and desulfurization. The exemplary hydrotreated stream 222 is fed to a fluid catalytic cracking (FCC) unit 226. In an exemplary embodiment, the FCC unit 226 is run under severe FCC conditions. An exemplary fluid catalytic cracking unit 226 is operated to form a selected fraction of hydrocarbons, such as hydrocarbons having carbon chain lengths of from six to ten, i.e., C6-C10 hydrocarbons. As a result, a cracked stream 228, for example an FCC C6-C10 hydrocarbon stream 228, is formed by the FCC unit 226. Under severe FCC processing, the aromatic content of the cracked stream may be as high as about 50 weight percent (wt %) to about 70 wt %. Other fractions formed by the FCC unit 226 are not illustrated but may include a C5⁻ stream or streams and a C11⁺ stream or streams.

The cracked stream 228 is fed to a selective hydrotreating unit 230, in an embodiment. The selective hydrotreating unit 230 saturates diolefins in the cracked stream 228. Further, the selective hydrotreating unit 230 converts mercaptans in the cracked stream 228 to disulfide compounds. Exemplary selective hydrotreating conditions include a temperature of about 250° C. to about 350° C. and a pressure of about 1000 kilopascals (kPa) to about 4000 kPa. As a result of the selective hydrotreating process, a hydrotreated stream 232 is formed with a reduced diolefin and mercaptan content.

In FIG. 1, the hydrotreated stream 232 is fed to a desulfurization unit 236. The desulfurization unit 236 removes the disulfide compounds from the hydrotreated stream 232 and forms a desulfurized stream 238. An exemplary desulfurized stream 238 has a sulfur content of less than 100 weight parts per million (wppm), such as about 10 to about 75 wppm. The exemplary embodiment feeds the desulfurized stream 238 to an aromatics extraction unit 240. The aromatics extraction unit 240 removes aromatics as an extract stream 242 from the remaining paraffins and olefins that form a raffinate stream 244. Typically, aromatics cannot be directly recovered at high purity by conventional distillation because of the close boiling components and azeotropes that form with aromatics. Therefore, they are typically recovered by extraction with a selective solvent. This can be accomplished through liquid-liquid extraction or by extractive distillation. An exemplary aromatics extraction unit 240 is an extractive distillation unit. An exemplary raffinate stream 244 primarily contains C6-C7 paraffins and olefins, such as greater than about 80%, greater than about 90%, or greater than about 95%, paraffins and olefins. In the exemplary embodiment of FIG. 1, the raffinate stream 244 is used in gasoline blending.

As shown in FIG. 1, stream 210 is first processed by a naphtha hydrotreating unit 250 to form a hydrotreated stream 252. The naphtha hydrotreating unit 250 may be used to prepare the C6-C10 cut of naphtha in stream 210 for downstream reforming with sensitive noble metal catalyst systems. In an exemplary process, the stream 210 is brought into the naphtha hydrotreating unit 250, mixed with hydrogen, and heated to a reaction temperature over a catalyst. Exemplary catalysts include nickel, molybdenum and compounds thereof. Exemplary reaction temperatures are from about 250° C. to about 400° C. The catalytic reaction converts the contaminants of noble metal catalyst systems, such as sulfur, nitrogen, oxygenates, via hydrogenolysis reactions to hydrogen sulfide, ammonia, and water so that they can be removed from the naphtha stream. Metals in the naphtha may be removed by adsorption onto the catalyst. Low levels of olefins or trace diolefins are saturated.

The resulting hydrotreated stream 252 contains paraffins, olefins and naphthenes and is fed to a reforming unit 256 for their conversion into aromatics. An exemplary reforming unit 256 is a catalytic reforming unit with continuous catalyst regeneration (CCR). The reforming unit 256 may be operated at a temperature of from about 495° C. to about 560° C. Compounds in the hydrotreated stream 252 are reformed to produce a reformate stream 260. Specifically, naphthenes are dehydrogenated to form aromatics, normal paraffins are isomerized to form isoparaffins, and paraffins are dehydrocyclized, i.e., dehydrogenated and aromatized, to form aromatics. Further, the aromatics present in the hydrotreated stream 252 can undergo demethylation and dealkylation reactions.

In the exemplary embodiment, the reformate stream 260 is fed to an aromatics complex 261, and specifically to a reformate splitter distillation column 262 therein. The reformate splitter distillation column 262 functions to separate or “split” the reformate stream 260 by distilling the reformate stream 260 into a heavier higher boiling fraction as stream 264 and a lighter, lower boiling fraction as stream 266. The reformate splitter distillation column 262 may be configured such that, for example, the heavier fraction in stream 264 includes primarily, such as greater than about 80%, greater than about 90%, or greater than about 95%, hydrocarbons having eight or more carbon atoms (C8⁺). The lighter fraction in stream 266 may include primarily (such as greater than about 80%, greater than about 90%, or greater than about 95%) hydrocarbons having seven or fewer carbon atoms (C7⁻).

The lighter fraction 266 is passed from the reformate splitter distillation column 262 to an extractive distillation process unit 270 for removing non-aromatic compounds from the lighter fraction 266. In one particular embodiment, extractive distillation process unit 270 may employ a sulfolane solvent to separate aromatic compounds from non-aromatic compounds. Other extraction methods, such as liquid-liquid solvent extraction are also well-known and practiced for separation of non-aromatic compounds from aromatic compounds, and their use in place of, or in addition to, extractive distillation process unit 270 is contemplated herein. Extractive distillation process unit 270 produces a raffinate stream 274 that includes primarily, such as greater than about 80%, greater than about 90%, or greater than about 95%, non-aromatic C7⁻ hydrocarbons and an extract stream 272 that includes primarily, such as greater than about 80%, greater than about 90%, or greater than about 95%, benzene and toluene. In FIG. 1, the raffinate stream 274 may be used in gasoline blending

In FIG. 1, the extract stream 242 formed by the aromatics extraction unit 240 may be fed through line 276 to a guard bed 280 that removes contaminants, such as remaining sulfur compounds or nitrogen compounds, from the extract stream 242 and forms stream 282 of aromatics. As shown, stream 282 is fed to the aromatics complex or processing zone 261 and is combined with extract stream 272 for processing in the aromatics complex including processing in a benzene distillation column 286, a toluene distillation column 288, a heavy aromatic distillation column 290, a xylene distillation column 292, a para xylene unit 294, a xylene isomerization unit 296, a light distillation unit 298, and a Tatoray process unit 300.

A fractionation process is performed on the streams 272 and 282 in the benzene distillation column 286 and benzene, having a lower boiling point than toluene, is removed from benzene distillation column 286 as a product stream 310. Toluene, having a higher boiling point than benzene, is removed from distillation column 286 as stream 312. Stream 312 may further include heavier aromatic hydrocarbons such as various xylene isomers. Stream 312 is fed to the toluene distillation column 288.

In the toluene distillation column 288, toluene is separated from heavier components, i.e., components having lower boiling points than toluene, and is removed as stream 314, such as overhead stream 314. The heavier aromatic hydrocarbons are removed as stream 316, such as bottom stream 316. As shown, the toluene rich stream 314 is fed to the Tatoray process unit 300. The Tatoray process unit 300 converts toluene into benzene and xylenes in a toluene disproportionation process. Further, the Tatoray process unit 300 converts a mixture of toluene and aromatic hydrocarbons having nine carbon atoms (C9) into xylenes in a transalkylation process. Hydrogen is fed to the Tatoray process unit 300 so that the disproportionation and transalkylation processes are conducted in a hydrogen atmosphere to minimize coke formation. As shown, a stream 318 of benzene, toluene and xylenes exits the Tatoray process unit 300 and is recycled to the benzene distillation column 286 for further processing.

Stream 316, including a mixture of xylenes, exits the toluene distillation column 288 and is fed to a para-xylene separation unit 294. Separation of para-xylene from the other xylenes in the para-xylene separation unit 294 results in the formation of an extract stream 319 containing para-xylene. A raffinate stream 320 is fed to the xylene isomerization unit 296 which reestablishes an equilibrium mixture of isomers via xylene isomerization and conversion of ethyl benzene to benzene or xylenes. The isomerized effluent 322 formed by the xylene isomerization unit 296 is fed to the light distillation unit 298, which forms a stream 324, such as overhead stream 324, primarily containing benzene, toluene, and ethylbenzene, and a stream 326, such as bottom stream 326, containing C8⁺ aromatics including primarily ortho-, meta-, para-xylenes. Stream 326 is combined with the C8⁺ fraction 264 from the reformate splitter distillation column 262 and is fed to the xylene distillation column 292. As shown, the xylene distillation column 292 further receives a bottom raffinate stream 328 from the para-xylene separation unit 294.

The xylene distillation column 292 separates a stream 336, such as an overhead stream 336, containing xylenes. Stream 336 is combined with the heavier aromatic hydrocarbons in stream 316 from the toluene distillation column 288 and is fed to the para-xylene separation unit 294. A stream 340, such as a bottom stream 340, including heavier components is removed from the xylene distillation column 292 and is fed to the heavy aromatic distillation column 290. The heavy aromatic distillation column 290 removes any lighter aromatics present in stream 340 as a stream 344, such as overhead stream 344. Stream 344 is combined with the toluene in stream 314 and is fed to the Tatoray process unit 300. Heavy aromatics are removed from the process in a stream 350, such as a bottom stream 350.

In the exemplary embodiment of FIG. 1 described above, the aromatics in the extract stream 242 removed from the FCC C6-C10 fraction in the aromatics extraction unit 240 are sent directly to the aromatics complex and do not undergo processing in the reforming unit 256. As a result, as compared to conventional processing in which aromatics are passed through the reforming unit 256, the flow rate to the reforming unit is reduced, the catalyst volume in the reforming reactors is reduced, the hydrogen requirement is reduced, and more para-xylene is produced in the aromatics complex. Para-xylene production is increased because the methyl groups from the extracted aromatics are conserved and the aromatics avoid dealkylation in the reforming unit, resulting in a higher methyl/phenyl ratio and higher para-xylene production. Further, in the exemplary embodiment an increased proportion of the olefinic FCC raffinate stream 244 is retained for use in gasoline blending in comparison to conventional processing. As a result, gasoline blending may attain high octane products without, or with only limited, addition of methyl tertiary butyl ether (MTBE) to the gasoline blend.

While the extract stream 242 removed from the aromatics extraction unit 240 is described above as being fed to the aromatics complex via line 276, in other embodiments the extract stream 242, or a portion of the extract stream 242 may be combined with the naphtha stream 210 upstream of the reforming unit 256 via line 354. As shown, line 354 delivers aromatics from extract stream 242 to the naphtha stream 210 upstream of the naphtha hydrotreating unit 250. In comparison, the line 360 delivers the desulfurized stream 238 of FCC C6-C10 hydrocarbon without having aromatics extracted therefrom. As a result, a method or apparatus using line 360 would not obtain as high a yield of aromatics.

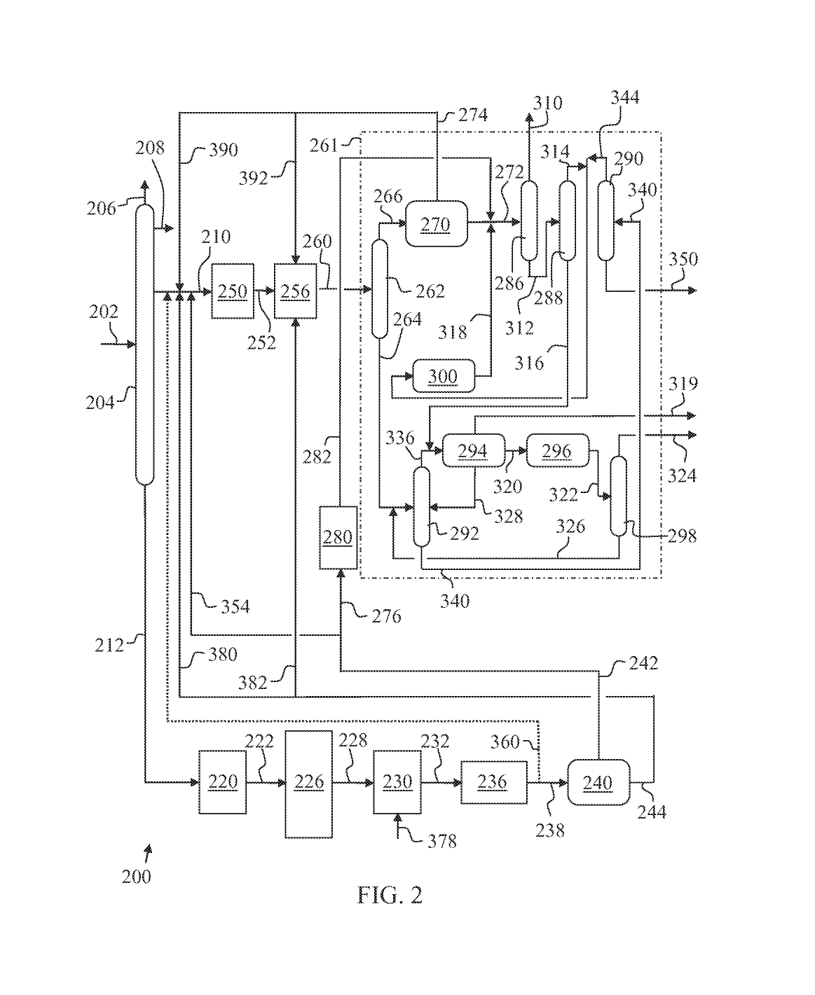

Referring to FIG. 2, an alternate embodiment of apparatus 200 is provided for processing hydrocarbons. In FIG. 2, the cracked stream 228 formed by cracking the C11⁺ hydrocarbon stream 212 is hydrotreated by a hydrotreating unit 230 that receives an optional stream of pyrolysis gas 378. An exemplary pyrolysis gas 378 contains C5-C12 hydrocarbons, such as C5-C9 hydrocarbons, including benzene, toluene, xylenes, olefins and dienes, and small amounts of contaminants, such as about 50 to about 500 wppm sulfur and about 0 to about 10 wppm nitrogen. The hydrotreating unit 230 is operated at mild conditions such as a temperature of from about 120° C. to about 180° C. and a pressure of from about 2750 kPa to about 3100 kPa (about 400 psia to about 450 psia). As a result of the selective hydrotreating process, a mild hydrotreated stream 232 is formed. In FIG. 2, the hydrotreated stream 232 is fed to a desulfurization unit 236. The desulfurization unit 236 removes the disulfide compounds from the hydrotreated stream 232 and forms a desulfurized stream 238. The desulfurized stream 238 is fed to an aromatics extraction unit 240. The aromatics extraction unit 240 removes aromatics as an extract stream 242 from the remaining paraffins and olefins that form a raffinate stream 244. In an exemplary embodiment, the raffinate stream 244 is primarily formed by C6-C7 paraffins and olefins, such as greater than about 80%, greater than about 90%, or greater than about 95%, C6-C7 paraffins and olefins

In the exemplary embodiment of FIG. 2, the raffinate stream 244 is fed to the reforming unit 256 or combined with the naphtha stream 210 upstream of the reforming unit 256. Specifically, the raffinate stream 244 may be fed through line 380 and combined with the naphtha stream 210 upstream of the naphtha hydrotreater 250. Alternatively, the raffinate stream 244 may be fed to the reforming unit 256 through line 382. In an exemplary embodiment, the raffinate stream 244 includes olefins and sulfur compounds that require hydrotreating before reforming Therefore, the raffinate stream 244 is fed to stream 210 upstream of the naphtha hydrotreater 250. In each of these embodiments, the paraffins and olefins in the raffinate stream 244 may be reformed into aromatics in the reforming unit 256.

As with the exemplary embodiment illustrated in FIG. 1, the reforming unit 256 of FIG. 2 forms a reformate stream 260. The reformate stream 260 is split by the reformate splitter distillation column 262 into a heavier, higher boiling fraction as stream 264 and a lighter, lower boiling fraction as stream 266. The lighter fraction in stream 266 is fed to an extraction unit 270, for example a solvent extraction unit such as a sulfolane unit. The extraction unit 270 removes aromatics in an extract stream 272 while forming a raffinate stream 274 including C6-C7 paraffins and olefins. In FIG. 2, the raffinate stream 274 is recycled back to either the stream 210 or to the reforming unit 256 for reforming with the hydrotreated naphtha stream 252. Specifically, the raffinate stream 274 may be fed via line 390 and combined with the naphtha stream 210 upstream of the naphtha hydrotreater 250. Alternatively, the raffinate stream 274 may be fed to the reforming unit 256 through line 392. In an exemplary embodiment, the raffinate stream 274 includes olefins and sulfur compounds that require hydrotreating before reforming Therefore, the raffinate stream 274 is fed to stream 210 upstream of the naphtha hydrotreater 250. In each of these embodiments, the paraffins and olefins in the raffinate stream 274 may be reformed into aromatics in the reforming unit 256.

The apparatus 200 of FIG. 2 removes aromatics from the cracked stream 228 before reforming the remaining portion (raffinate 244) of the cracked stream 228. As a result, production of aromatics from the cracked stream 228, and thus from the feedstock 202, is enhanced. Further, non-aromatics isolated in the raffinate stream 244 and in the reformate raffinate 274 are passed through the naphtha reforming unit 256 to further increase aromatic content of the reformate stream 260. As shown, the line 360 delivers the cracked hydrocarbon stream 228 to the naphtha stream 210 (after hydrotreating and desulfurization) for reforming without having aromatics extracted therefrom. As a result, a method or apparatus using line 360 would not obtain as high a yield of aromatics as the embodiment of FIG. 2.

As described herein, methods and apparatuses for processing hydrocarbons have been provided. In an exemplary embodiment, a method and apparatus extracts aromatics from a cracked hydrocarbon stream, extracts aromatics from a naphtha reformate and processes the aromatics to form selected aromatic product streams. Aromatics extracted from the cracked hydrocarbon stream do not pass through a naphtha reforming unit and avoid dealkylation therein.

While at least one exemplary embodiment has been presented in the foregoing detailed description, it should be appreciated that a vast number of variations exist. It should also be appreciated that the exemplary embodiment or exemplary embodiments are only examples, and are not intended to limit the scope, applicability, or configuration of the claimed subject matter in any way. Rather, the foregoing detailed description will provide those skilled in the art with a convenient road map for implementing an exemplary embodiment or embodiments. It being understood that various changes may be made in the function and arrangement of elements described in an exemplary embodiment without departing from the scope set forth in the appended claims.

What is claimed is:

1. A method for processing hydrocarbons, the method comprising the steps of:
fractionating a feed stock to form a C6-C10 naphtha stream and a C11⁺ stream;
reforming the C6-C10 naphtha stream;
cracking the C11⁺ stream to form a stream of C6-C10 hydrocarbons;
extracting aromatics from the stream of C6-C10 hydrocarbons to form an extract stream;
combining the C6-C10 naphtha stream and the extract stream containing the aromatics; and
processing the C6-C10 naphtha stream and the extract stream in an aromatics complex to form selected aromatic products.

2. The method of claim 1 comprising combining the C6-C10 naphtha stream and the extract stream containing the aromatics after reforming the C6-C10 naphtha stream.

3. The method of claim 1 comprising combining the C6-C10 naphtha stream and the extract stream containing the aromatics before reforming the C6-C10 naphtha stream, and wherein reforming the C6-C10 naphtha stream comprises reforming the C6-C10 naphtha stream and the extract stream containing the aromatics.

4. The method of claim 1 further comprising hydrotreating the C6-C10 naphtha stream before reforming the C6-C10 naphtha stream.

5. The method of claim 1 further comprising:
selectively hydrotreating the stream of C6-C10 hydrocarbons to form a hydrotreated stream; and
desulfurizing the hydrotreated stream to form a desulfurized stream, wherein extracting aromatics from the stream of C6-C10 hydrocarbons comprises extracting aromatics from the desulfurized stream.

6. The method of claim 1 wherein extracting aromatics from the stream of C6-C10 hydrocarbons forms a raffinate stream containing olefins.

7. The method of claim 1 wherein extracting aromatics from the stream of C6-C10 hydrocarbons forms a first raffinate stream containing olefins, wherein reforming the C6-C10 naphtha stream comprises forming a reformate, and wherein the method further comprises:
extracting aromatics from the reformate and forming second raffinate stream; and
feeding the first raffinate stream and the second raffinate stream to gasoline blending.

8. The method of claim 1 further comprising:
combining the stream of C6-C10 hydrocarbons with pyrolysis gas; and
hydrotreating the stream of C6-C10 hydrocarbons and the pyrolysis gas to form a hydrotreated stream, wherein extracting aromatics from the stream of C6-C10 hydrocarbons comprises extracting aromatics from the hydrotreated stream.

9. The method of claim 1 wherein extracting aromatics from the stream of C6-C10 hydrocarbons to form an extract stream comprises forming a raffinate stream and wherein the method further comprises combining the raffinate stream with the C6-C10 naphtha stream.

10. The method of claim 1 wherein:
extracting aromatics from the stream of C6-C10 hydrocarbons to form an extract stream comprises forming a raffinate stream;
reforming the C6-C10 naphtha stream comprises forming a reformate;
the method further comprises extracting aromatics from the reformate and forming a reformate raffinate stream; and
reforming the C6-C10 naphtha stream comprises reforming the C6-C10 naphtha stream and the reformate raffinate stream.

11. A method for processing hydrocarbons, the method comprising the steps of:
fractionating a hydrocarbon stream in a fractionation unit and forming a first fraction and a second fraction;
introducing the first fraction to a reforming unit and reforming the first fraction to form a reformate stream;
feeding the reformate stream to an aromatics processing zone and producing a benzene product and a para-xylene product therefrom;
introducing the second fraction into a fluid catalytic cracking (FCC) unit and cracking the second fraction to form a cracked stream of hydrocarbons;
feeding the cracked stream to an aromatic extraction unit and extracting aromatics from the cracked stream in an extract stream;
bypassing the reforming unit with the extract stream and introducing the extract stream to the aromatics processing zone, wherein the extract stream is processed in the aromatics processing zone to produce the benzene product and the para-xylene product.

12. The method of claim 11 further comprising:
extracting aromatics from the reformate stream in a reformate extract stream; and
combining the extract stream and the reformate extract stream, wherein producing a benzene product and a para-xylene product comprises processing the extract stream and the reformate extract stream.

13. The method of claim 11 wherein extracting aromatics from the cracked stream in an extract stream comprises forming a raffinate stream containing olefins, and wherein the method further comprises combining the raffinate stream with the first fraction before introducing the first fraction to a reforming unit.

14. The method of claim 11 wherein extracting aromatics from the cracked stream in an extract stream comprises forming a first raffinate stream containing olefins, and wherein the method further comprises:
extracting aromatics from the reformate stream in a reformate extract stream and forming a second raffinate stream; and
combining the first raffinate stream and the second raffinate stream with the first fraction before introducing the first fraction to a reforming unit.

15. The method of claim 11 wherein:
extracting aromatics from the cracked stream in an extract stream comprises forming a raffinate stream containing olefins; and
the method further comprises reforming the raffinate stream in the reforming unit.

16. The method of claim 11 wherein:
extracting aromatics from the cracked stream in an extract stream comprises forming a first raffinate stream containing olefins; and wherein the method further comprises:
extracting aromatics from the reformate stream in a reformate extract stream and forming a second raffinate stream; and
feeding the first raffinate stream and the second raffinate stream to the reforming unit.

17. The method of claim 11 further comprising hydrotreating the cracked stream with a pyrolysis gas to form a hydrotreated stream, wherein feeding the cracked stream to an aromatic extraction unit and extracting aromatics from the cracked stream in an extract stream comprises feeding the hydrotreated stream to an aromatic extraction unit and extracting aromatics from the hydrotreated stream in an extract stream.

18. The method of claim 11 wherein cracking the second fraction to form a cracked stream of hydrocarbons having a selected carbon chain length comprises cracking the second fraction to form a stream of C6-C10 hydrocarbons.

19. The method of claim 11 wherein extracting aromatics from the cracked stream in an extract stream forms a first raffinate stream containing olefins, and wherein the method further comprises:
extracting aromatics from the reformate stream and forming second raffinate stream; and
feeding the first raffinate stream and the second raffinate stream to gasoline blending.

20. An apparatus for processing hydrocarbons comprising:
a fractionation unit configured to form a C6-C10 naphtha stream and a C11⁺ stream from a feed stock;
a cracking unit configured to crack the C11⁺ stream to form a stream of C6-C10 hydrocarbons;
a first aromatic extraction unit configured to extract a first aromatic stream from the stream of C6-C10 hydrocarbons;
a reforming unit configured to reform the C6-C10 naphtha stream and form a reformate;
a second aromatic extraction unit configured to extract a second aromatic stream from the reformate; and
an aromatics processing unit configured to produce a benzene product and a para-xylene product from the first aromatic stream and the second aromatic stream.
